(19) United States
(12) Patent Application Publication
(10) Pub. No.: US 2011/0206735 A1
(43) Pub. Date: Aug. 25, 2011

(54) Tumorigenic CancerStemCells, Methods of Isolating and Using the Same

(76) Inventor: Yong Qian, San Diego, CA (US)

(21) Appl. No.: 13/034,580

(22) Filed: Feb. 24, 2011

Related U.S. Application Data
(60) Provisional application No. 61/338,898, filed on Feb. 24, 2010.

(51) Int. Cl. A61K 39/00; C12N 5/095; A61P 35/00

(52) U.S. Cl. 424/277.1; 435/354

(57) ABSTRACT

The present invention provides an enriched preparation of cancer stem cells selected by a process that eliminates non-cancer stem cells under extreme culture conditions. The cancer stem cells of the invention are tumorigenic in vivo and form in vitro multilayered 3D-tumors attached to culture plates. The present invention also provides a whole-cell cancer vaccine, which is able to eradicate all cancer cells to make cancer-free mice in a xenograft tumor model. The cancer stem cells of the invention are very useful for basic cancer research, and development of therapeutic and preventive agents for various cancer types.

### CROSS-REFERENCE TO RELATED APPLICATIONS

This application claims benefit of priority to U.S. provisional application Ser. No. 61/338,898, filed Feb. 24, 2010, the contents of which are incorporated by reference herein.

### BACKGROUND OF THE INVENTION

1. Field of the Invention

The present invention relates generally to preparations and applications of stem cells, and in particular, relates to preparations and applications of cancer stem cells from cancer cell lines, tumors, or blood samples with cancer cells.

2. Description of the Related Art

Due to limited angiogenesis and vasculature, limited space and great stress including hypoxia and limited nutrients caused very crowded cancer cells resulted from uncontrolled cancer cell division and growth, cancer cells located in the middle of the tumor may die due to starvation, necrosis, apoptosis and other unknown mechanisms. However, some of them may mutate and survive under those extreme conditions. When the environment improves, the survived cancer cells form a new tumor again due to their tumorigenic capability. These mutants are hard-to-kill cancer stem cells that can tolerate stress and apoptosis inducers including anti-cancer agents. These cancer stem cells are not only tumorigenic but also heterogeneous and mutating under the immune screening pressure. The tumorigenic, heterogeneous and mutating characteristics or capacity are genetically built in cancer stem cells' genomes. When these tumorigenic cancer stem cells grow in vitro with complete medium without stress or immune screening, they tend to be less mutating, less heterogeneous and less tumorigenic over time and over generations since there is no selection pressure in the growing environment. Thus, cancer cell lines have a very small percentage, 0 to 5%, of cancer stem cells. There is a great need to inactivating none cancer stem cells and concentrating cancer stem cells.

Many anti-cancer agents are not effective against malignant tumor since they are not effective against cancer stem cells that are resistant to apoptosis inducers and viable under extreme conditions including hypoxia and depleted nutrients in the middle of a tumor. These cancer stem cells are the “seeds” for tumor recurrence and metastasis after chemotherapy and radiotherapy. In order to develop more effective anti-cancer agents targeting the whole population of tumor cells, including cancer stem cells, there is a need to generate and enrich cancer stem cells in vitro that can match the tumorigenic capacity of cancer stem cells in vivo.

Biomarkers, including CD133, CD131, CD20 and others, have been used to isolate cancer stem cells. However, a large body of evidence has found that 1) cancer stem cells isolated by biomarker(s) may be a subpopulation of cancer stem cells favorably selected by their biomarker(s), while other subpopulations of cancer stem cells were unfairly leaving out or unselected since they do not have the biomarker(s). There is a great need to select cancer stem cells based on tumorigenic capacity rather than biomarkers.

Cancer cell lines may have clones or sub-clones of fixed mutations, mature cancer cells and a very small number of cancer stem cells (0 to 5%). Since the clones or sub-clones of fixed mutations and mature cancer cells are the majority in the cell population of cancer cell lines, cancer research results using these cancer cell lines may target clones or sub-clones of fixed mutations and mature cancer cells but not cancer stem cells. In order to develop cures for cancer, cancer stem cells are needed for cancer research with potential clinical application.

When cancer cells from a cancer cell line is used to make a whole-cell cancer vaccine (1,2), antigens in sub-clones of fixed mutations and mature cancer cells are over represented but antigens in cancer stem cells are barely or not represented at all. Cancer stem cells are needed to make better whole cell cancer vaccines that may induce complete immune responses to kill all cancer cells, including pre-cancer cells or cancer progenitor cells, cancer stem cells, mature cancer cells and metastasized cancer cells.

Anti-cancer agents are routinely screened and tested using cancer cells from a cancer cell line or cancer cell lines, with 0 to 5% cancer stem cells. Thus, these anti-cancer agents are effective against clones or sub-clones of fixed mutants and mature cancer cells but may not be effective against cancer stem cells. Cancer stem cells are one of the major reasons for tumor recurring after a cancer treatment including chemotherapy, radiotherapy and immunotherapy. There is a great need in using cancer stem cells for more effective anti-cancer agent screening and development.

### SUMMARY OF THE INVENTION

The present invention provides an enriched preparation of cancer stem cells, the method to make the same, and applications to use the same. Cancer stem cells, or “CancerStemCells” as used throughout the application, refer to tumor-initiating cells that have extensive proliferative potential and the ability to give rise to a tumor in vivo and/or in vitro. CancerStemCells are reported to be resistant to harsh growth conditions such as low nutrients and low oxygen levels. The present invention provides a method of selecting cancer stem cells based on their ability to sustain extreme growth conditions and the ability to form in vitro 3D-tumors. In some embodiments of the invention, CancerStemCells are enriched from cancer cells obtained from a cell line, a tumor, a bone marrow sample, or a blood sample. CancerStemCells generally represent less than 5% of a population of cancer cells from a cell line, a tumor, a bone marrow sample, or a blood sample from cancer patients. To isolate CancerStemCells, a large population of cancer cells are cultured under one or a combination of extreme culture conditions to selectively kill the non-CancerStemCells. The extreme culture conditions are selected so that the majority of cells in the population, non-CancerStemCells, will die before CancerStemCells. The CancerStemCells can be enriched and selected after the death phase of non-CancerStemCells. Different from the non-CancerStemCells which grow to a mono-layer of cells, CancerStemCells form in vitro 3D-tumors, islands of multilayered cells, attached to the bottom of collagen-coated plates. In some embodiments of the invention, the sphere-forming cells are at least 20%, 30%, 40%, 50%, 60%, 70%, 80%, 90% or up to 100% of the total population of the CancerStemCell preparation. In most of the CancerStemCell preparations, the sphere-forming cells represent more than 80% of the total population of the cells. In one embodiment of the invention, a preparation of CancerStemCells is made from a population of the sphere-forming cells. In another embodiment of the invention, a CancerStemCell cell line is made by expanding a single 3D-tumor.

In one embodiment of the invention, the extreme culture conditions include irradiation, cytotoxic drug treatment, low nutrient level/starvation, low oxygen level/hypoxia, and low carbon dioxide level. In a preferred embodiment of the invention, cancer cells are cultured in a medium with depleted nutrients analogous to in vivo cell growth in tumor region with limited nutrient supply. In some embodiments of the invention, the enriched CancerStemCells are highly tumorigenic when injected into nude or wild type mice. For example, as low as 300 CancerStemCells sub-Q injected into a mouse can generate palpable tumors in the animal, whereas one million unselected cancer cells are generally required to generate tumors in a mouse.

In some embodiments of the invention, CancerStemCells selected from different selection processes and/or different cell sources (e.g. cell lines, tumors, or blood samples) are pooled together to make a cancer-specific or animal-specific preparation.

In some embodiments of the invention, sphere-forming CancerStemCells can be further sorted into sub-groups based on the expression of different biomarkers.

The present invention provides a cancer vaccine derived of the CancerStemCells of the invention, which can be used to elicit immune responses to a broad spectrum of cancer-specific antigens in an individual for cancer treatment and cancer prevention. The CancerStemCells of the invention are highly proliferative, tumorigenic, heterogeneous, and hard-to-kill, characteristic of the “cancer seed cells” underlying tumor initiation, cancer chemotherapy and radiotherapy resistance, tumor metastasis, and tumor relapse. Specific eradication of CancerStemCells in cancer patients may hold the key to eradicate cancer by its root. CancerStemCells have accumulated a broad range of cancer-specific mutations and chromosome abnormalities that are shared with other cancer cells such as mature cancer cells, metastatic cancer cells, and pre-cancer cell/cancer progenitor cells. Therefore, cancer vaccines derived from the CancerStemCells of the invention are effective at eliciting immune responses targeted to not only cancer stem cells, but also mature cancer cells, metastatic cancer cells, pre-cancer cells, and cancer progenitor cells, whereas normal cells are spared from the immune system attack.

The method to make whole-cell cancer vaccines from cancer cells is described in patent application Ser. No. 12/322,237 and patent application Ser. No. 11/825,246, which is incorporated by reference herein. In some embodiments of the invention, a cancer vaccine is derived from CancerStemCells inactivated and made harmless by an appropriate process. The appropriate process to inactivate CancerStemCells will disable the CancerStemCells to grow or replicate while keep cell membranes intact. The inactivated CancerStemCells preserve the cancer-specific antigens inside the cell, but become harmless to an individual when injecting into the body. The inactivating process include, but not limited to, one or a combination of operations such as proteinase digestion, chemical treatment (e.g. formalin treatment and phenol treatment), physical treatment (e.g. heat and freeze-thaw-freeze treatment), and irradiation (e.g. y-ray, x-ray, microwave, and UV treatment). In one preferred embodiment of the invention, effective cancer vaccines are made from CancerStemCells using proteinase digestion. Proteinases are used to inactivate CancerStemCells by digesting extracellular proteins and extracellular portion of membrane proteins while keep the cell membrane intact. For example, cancer vaccines derived from CancerStemCells by Tumorase™ (BioMedicure, San Diego, Calif.) digestion were effective at reducing tumor size and delaying tumor formation in a xenograft mice model. Remarkably, five out of ten vaccinated mice were cancer free for their lifetime without any noticeable side effects.

In some embodiments of the invention, cancer vaccines are derived from human CancerStemCells selected from a patient's tumor cells. The cancer vaccine can be used to treat the same patient to elicit immune responses to eradicate cancer cells. In some embodiments of the invention, cancer vaccines are derived from cancer type-specific CancerStemCells. In another embodiment of the invention, cancer vaccines are derived from a combined preparation of human CancerStemCells selected and enriched from all of the known human cancer types, thus making a pan-cancer vaccine targeting multiple human cancers. The antigen species in the pan-cancer vaccine may be diversified enough to potentially cover all antigen species in a tumor. It is speculated that this pan-cancer vaccine can be used as a therapeutic as well as a preventive agent against different cancers.

In some aspects, a screening method is provided for identifying candidate anti-CancerStemCell therapeutic agents. In some embodiments of the invention, the method comprises screening a chemical compound library for candidate therapeutic agents that inhibit, kill, or promote differentiation of the CancerStemCells of the invention. In some embodiments of the invention, the method comprises screening a chemical compound library for therapeutic agents that selectively kill the CancerStemCells, but spare the normal cells. In some embodiments of the invention, the method may be used to screen for therapeutic agents custom tailored to a specific patient. The method in this application would comprise screening for therapeutic agents that selectively kill the CancerStemCells but not normal stem cells isolated from the identified patent.

Furthermore, the enriched CancerStemCells are very useful for basic cancer research, including cancer cell population genetics or cancer stem cell population genetics, cancer cell population cytogenetics or cancer stem cell cytogenetics, cancer cell population biology and molecular biology or cancer stem cell biology and molecular biology, cancer cell population immunology or cancer stem cell immunology, cancer cell population pharmacology or cancer stem cell pharmacology. Enriched CancerStemCells are very useful for anticancer agent screening, development and diagnostics as well.

These and other objects, advantages, and features of the invention will be better understood by references to the drawings, figures, photos and the detailed description of the invention.

### BRIEF DESCRIPTION OF THE DRAWINGS

For a better understanding of the present invention and to show more clearly how the same may be carried into effect, references are made, by example, to the accompanying drawings.

FIG. 1. Flow chart of selecting CancerStemCells from cultured cancer cells.

FIG. 2. A Cell Growth, Death and Survival Curve.

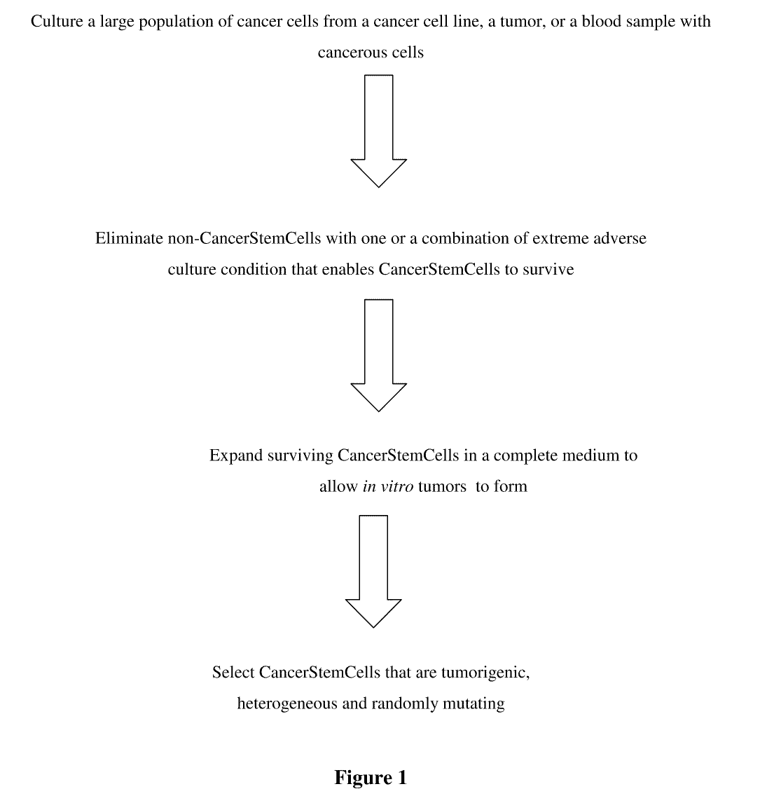

Cells cultured in limited nutrients normally undergo four phases: lag phase, log phase, stationary phase, and death phase. CancerStemCells that can survive extreme adverse culturing condition have an additional phase after death phase: survival phase.

The lag phase is the time when cells adjust to the new environment before active division and growth. The log phase is the time during which cells are actively dividing and growing when the nutrients are abundantly available. The stationary phase is the time when nutrients became limited and the number of dividing and growing cells almost equal to the number of cells dying. The death phase is the time period when nutrients become very limited or exhausted. During this phase, a large number of cells are dying rapidly and dead cells also secret apoptosis inducers that further accelerate cell death. Cancer stem cells can tolerate adverse extreme conditions such as limited nutrients and are resistant to apoptosis inducers. Cancer stem cells can survive the dead phase and enter the cancer stem cell survival phase which could last from several days to several weeks. This is the key discovery of this invention.

FIG. 3. in vitro tumor formed by CancerStemCells (CSC-016) expanding in a complete medium.

3A, After the selection process, CancerStemCells survived the extreme culture condition were expanded in Leibovitz's L15 medium with serum. CancerStemCells formed multi-layered 3D in vitro tumor structures instead of a mono-layer of cells in the culture plate.

3B, A schematic picture of an in vitro 3D-tumor formed in a culture dish.

FIG. 4, Comparison of tumor formation in unvaccinated mice and mice vaccinated with a cancer vaccine made from CancerStemCells.

Upper Panel: vaccinated mice group. Mice in this group were vaccinated with 300,000 cancer vaccine liposomes made from mouse melanoma CancerStemCells on Day 1 and Day 15. Lower Panel: unvaccinated mice group. Mice in this group received no vaccine.

Ten mice in each group were sub-Q injected with 300,000 living melanoma CancerStemCells on Day 30. Ten out of ten Mice in the unvaccinated group developed large tumors (shown in the lower panel) and died within 25 days after CancerStemCell injection. All the ten vaccinated mice did not develop tumor within the first two months after the sub-Q injection. Five vaccinated mice eventually developed tumors between two and four months, and five out of ten vaccinated mice were cancer free for their lifetime. The five cancer free mice were shown at the upper panel.

FIG. 5. Process of combining multiple CancerStemCells to make a combined preparation

CancerStemCells from various selection processes are combined proportionately to further diversify the heterogeneity of a cancer stem cell population. n is the total number of CancerStemCell (CSC) samples selected from different selection processes. Different selection processes include using different extreme culturing conditions such as limited nutrients, low levels of O2 or CO2, irradiation, or treatment with cytotoxic agents. Different selection processes also include selection from different cell sources, for example, different cancer cell lines or different tumors.

### DETAILED DESCRIPTION

It is advantageous to define several terms before describing the invention. It should be appreciated that the following definitions are used throughout the application unless specified otherwise.

The term “normal cells” as used herein, refers to living cells with a normal genome without any mutations leading to the over-expression of any oncogenes, such as muc1, Ras, BCR-ABL and many others, or the loss of expression of any tumor suppressor genes, such as p53, p63, p73 and many others. Normal cells are characterized by their capability of forming a clone in vivo and in vitro, under the same growth condition. Individual cells within the same clone are not only genetically identical but also morphologically similar or the same. These normal cells are further characterized by their contact inhibition for growth and programmed cell death or apoptosis. When they are growing on an attachment plate, they form a mono-layer of cells attached to the plate.

The term “pre-cancer cells” or “progenitor cancer cells” as used herein, refers to living cells with mutations leading to either the over-expression of any oncogenes or the loss of any tumor suppressor genes, but not both. These cells are different from normal cells since their genomes have significant mutations that lead to either the over-expression of one to several oncogenes or the missing of one to a few tumor suppressor genes. However, they may be morphologically similar or the same to normal cells. They are on their way to further mutate to become cancer cells when pre-cancer cells have both the over-expression of oncogene(s) and the missing of tumor-suppressor gene(s) in the same cell. Either the over-expression of oncogenes or the missing of tumor suppressor genes are believed to be the driving force for further mutation leading to cancer cells. Pre-cancer cells are not cancer cells yet since their cell division and growth are not out of control yet and they may still have contact inhibition for growth and programmed cell death or apoptosis.

The term “cancer cells” as used herein, refers to any living cells with genetic mutations leading to the over-expression of at least one oncogene(s) and the loss of at least one tumor-suppressor gene(s) within the same cell. Cancer cells are characterized by their uncontrolled cell division, growth and loss of contact inhibition. Cancer cells may be different in shape, size and capability of tumorigeneity. According to developmental stages, cancer cells may be further classified into several subpopulations including mature cancer cells, cancer stem cells and metastasized cancer cells. The term “a population of cancer cells” as used herein, refers to a large number of heterogeneous cancer cells obtained from a cancer cell line, a tumor, a bone marrow sample, or a blood sample with cancer cells. The population of cells may include cancer stem cells, mature cancer cells, metastasized cancer cell, pre-cancer cells/progenitor cancer cells. Depending on the cell source, cancer stem cells may represent 0 to 5% of the population of the cancer cells.

The term “tumorigenic” as used herein, refers to a cell's ability to form a tumor in vivo and/or in vitro. The term “tumorigenic in vivo”, refers to a cell's ability to form a tumor inside an animal's body. The term “tumorigenic in vitro”, refers to a cell's ability to form an in vitro tumor in the cell culture. An in vivo tumor is a mass of irregularly arranged and inter-connected cells that undergo abnormal growth and uncontrolled division inside a body. Similar to in vivo tumor, in vitro tumor is a three-dimensional, multilayered mass of irregularly arranged and inter-connected cells formed in the cell culture. Tumor spheres formed by CancerStemCells of the invention in the culture are an example of in vitro tumors. Although called tumor spheres, these in vitro 3D-tumors are mostly shaped as a hemisphere, or other irregular shapes as they are attached to the bottom of the culture plate. Both in vivo and in vitro tumors are originated from a single cancer stem cell. Furthermore, in vitro tumorigenicity works in good parallel with in vivo tumorigenicity.

The term “CancerStemCells”, or “cancer stem cells” as used herein, refers to tumor-initiating cells that have extensive proliferative potential and can give rise to an in vivo tumor and/or in vitro tumor. These CancerStemCells possess ability to survive extreme growth conditions analogous to those in the middle of solid tumors with limited nutrients, O₂ and CO₂. The CancerStemCells are also more resistant to irradiation and cytotoxic agents than matured cancer cells. The CancerStemCells of the invention are selected based on the ability to sustain extreme culture conditions and the tumorigenic capability, not on the expression of stem cell biomarkers. Different from cancer stem cells selected by biomarkers which form floating tumor spheres, the CancerStemCells selected above form in vitro multilayered tumor spheres that are attached to culture plates. The CancerStemCells are a heterogeneous group of cancer cells that have tumorigenic capability built in the genetic mutations of the genome leading to over-expression of multiple oncogenes and loss of expression of multiple tumor suppressor genes in the same cells. The term “non-CancerStemCells” used therein, refers to cells other than the CancerStemCells selected above, which include matured cancer cells, pre-cancer cells, metastatic cancer cells, and other cell types in a population. In an unselected population of cancer cells, non-CancerStemCells are the majority of the cells. Compared to CancerStemCells, the non-CancerStemCells are more susceptible to extreme culture conditions and will die before CancerStemCells under the extreme culture conditions. Non-CancerStemCells usually grow to a mono-layer of cells in the culture.

The term “extreme culture conditions” as used herein, refers to harsh culture conditions that are more favorable to CancerStemCells than non-CancerStemCells, which can be used to separate CancerStemCells from the rest of cancer cells in a population. It is known that cancer stem cells are more likely to survive than other cancer cells in the middle of a solid tumor where nutrients, O₂ and CO₂ are limited. CancerStemCells are also more resistant to irradiation and cytotoxic agents. The extreme culture conditions for selecting CancerStemCells include, for example, culturing under limited nutrients, low levels of O₂ and/or CO₂, irradiation, and in the presence of cytotoxic agents. A preferred condition for selecting CancerStemCells is to culture a cancer cell population under a medium with limited nutrients, where non-CancerStemCells will die at the death phase due to lack of nutrients and CancerStemCells can survive the death phase and enter the survival phase.

The term “cancer vaccine” as used herein, refers to harmless variants or derivatives of cancer cells that preserve cancer specific antigens and can elicit immune responses against the cancer specific antigens. To make a whole-cell cancer vaccine, cancer cells are inactivated by a process that keeps the cell membrane of the cancer cells intact. The inactivating process include, but not limited to, one or a combination of operations such as proteinase digestion, chemical treatment (e.g. formalin treatment and phenol treatment), physical treatment (e.g. heat and freeze-thaw-freeze treatment), and irradiation (e.g. y-ray, x-ray, microwave, and UV treatment). Whole-cell cancer vaccine has the advantage of preserving a broad range of cancer specific antigens inside the cell membrane, allowing the immune system to attach cancer cells from multiple avenues. One problem associated with the application of whole-cell cancer vaccine is lack of immunogenicity, partially due to the expression of self-recognition proteins such as major histocompability complex (MHC) on the cell surface. Proteinase digestion of extracellular portions of membrane proteins like MHC inactivates the cancer cells and increases the immunogenicity of the whole-cell cancer vaccine, thus producing an effective cancer vaccine for cancer treatment.

The term “mature cancer cells” as used herein, refers to differentiated cancer cells that may be no longer tumorigenic but maintain limited proliferative potential. They are not randomly mutating although they are heterogeneous in subpopulations. Within a subpopulation, mature cancer cells may be clones or subclones with fixed mutations.

The term “metastasized cancer cells” as used herein, refers cancer cells that express immune recognition molecules including major histocompability complex II (MHC II). These cancer cells can move around in the blood and lymph stream without being recognized or attacked by the immune system.

Most methods for preparing cancer stem cells involve selection by particular biomarker(s), which introduces a selection bias towards the particular biomarker(s) and under-representation of cancer stem cells without the biomarkers(s). The present invention provides a method of selecting cancer stem cells based on their ability to sustain adverse growth conditions and the ability to form in vitro tumor spheres, which overcomes the biomarker selection bias. Because they are selected by tumorigenicity, not by any biomarkers, CancerStemCells of the invention are a mixed population of cancer stem cells, not favoring any subpopulation of cancer stem cells with specific biomarkers and not biased against any subpopulations without specific biomarkers either. This is very significant since using the CancerStemCells of the invention for research and drug screening is no longer biased toward certain subpopulations of cancer stem cells with biomarkers, enabling research results to address the whole population of cancer stem cells with greater clinical application potential, and anti-cancer drug screening to target all cancer stem cells in vivo. The antigen species in CancerStemCells may be diversified enough to potentially cover all antigen species in a tumor. Vaccination using whole cell cancer vaccines derived from CancerStemCells of the invention may induce immune responses to kill all cancer cells, including pre-cancer cells, cancer progenitor cells, cancer stem cells, mature cancer cells and metastasized cancer cells for a potential cure.

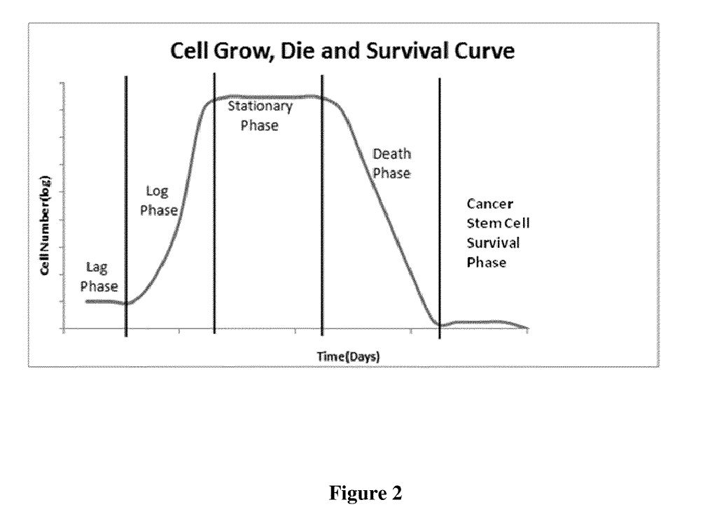

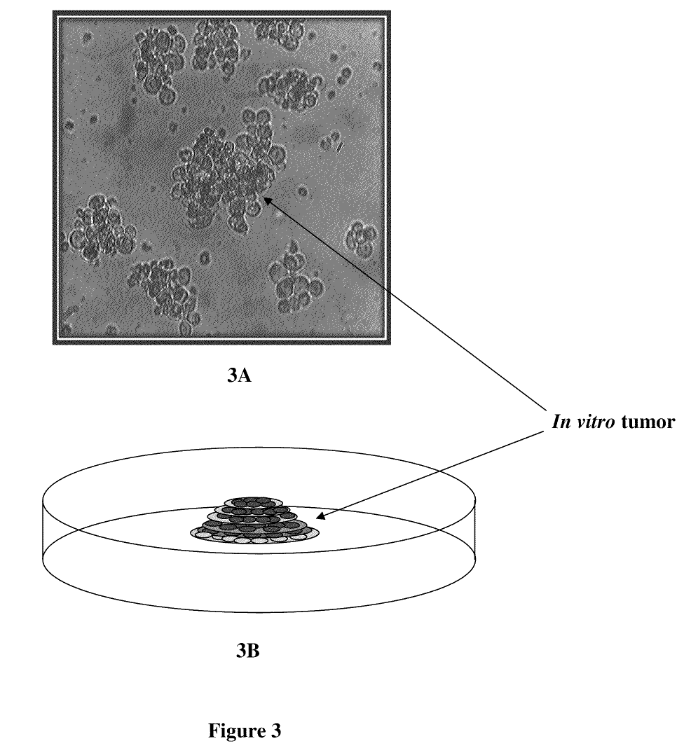

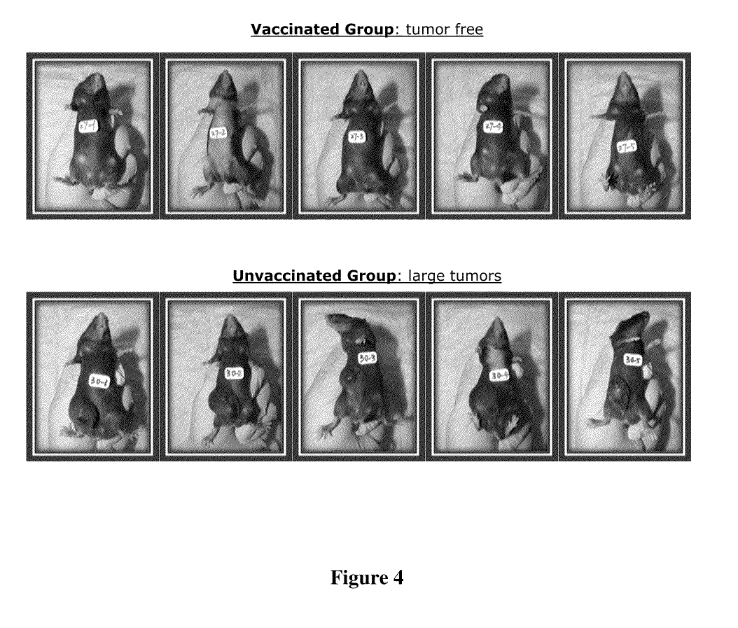

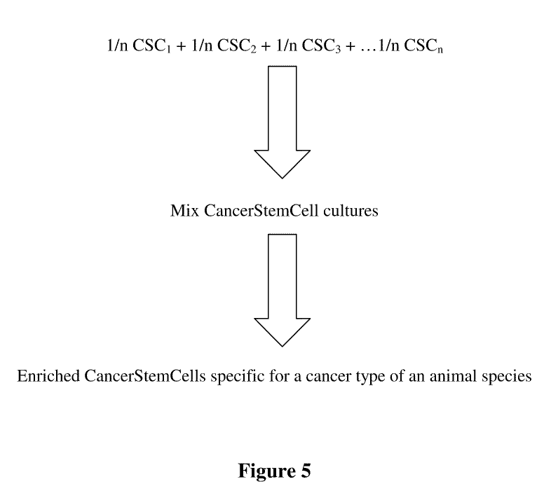

The present invention provides an enriched preparation of CancerStemCells selected from a population of cancer cells. In one embodiment of the invention, the enriched preparation of CancerStemCells is selected from the population of cancer cells by eliminating non-CancerStemCells in the population under extreme culture conditions. A large population of cancer cells is obtained from a cancer cell line, a tumor, a bone marrow sample or a blood sample with cancer cells. The unselected cells are a heterogeneous population which may include cancer stem cells, mature cancer cells, pre-cancer cells, metastasized cancer cells, even some normal cells disassociated from a tumor tissue. Depending on cell sources, CancerStemCells represent 0 to 5% of cells in the population. CancerStemCells are known to be more resistant to harsh conditions than other types of cancer cells and non-cancerous cells. Extreme culture conditions such as culturing under limited nutrients, low O₂ and CO₂ levels, irradiation, or in the presence of cytotoxic agents can be chosen as a selection pressure to eliminate non-CancerStemCells but spare CancerStemCells. Different culture conditions can be applied on a cancer cell population until an ideal selection condition is found. The ideal selection condition will lead to a cell growth curve as shown in FIG. 2 with five phases: lag phase, log phase, stationary phase, death phase, and CancerStemCell survival phase. For example, cancer cells growing in a medium with limited nutrients undergo the five phases as above.

In one embodiment of the invention, an enriched preparation of CancerStemCells is selected from a population of cancer cells by culturing the cancer cells in a medium with limited nutrients. Non-CancerStemCells are eliminated during the death phase when nutrients become depleted, whereas CancerStemCells can survive on limited nutrients and enter the survival phase. The surviving CancerStemCells are cultured in a complete medium with serum. In some embodiments of the invention, the surviving CancerStemCells form multilayered tumor spheres when cultured in a complete growth medium. Unlike cancer stem cells selected by biomarkers, which can only form floating tumor spheres in growth medium without serum, the tumor spheres formed by the CancerStemCells are attached to culture plates in a complete growth medium with serum. The attached tumor sphere has a three-dimensional structure with multiple layers of irregularly arranged and inter-connected cells, bearing much similarity to early tumor formation in the body. The sphere-forming cells have high in vivo tumorigenic potential when injecting into an animal. In some embodiments of the invention, the sphere-forming cells are at least 20%, 30%, 40%, 50%, 60%, 70%, 80%, 90%, 95%, or up to 100% of the total population of the CancerStemCells. When cultured with sufficient nutrients, the tumor spheres grow not in two-dimensional

In some embodiments of the invention, a single tumor sphere originated from a single CancerStemCell is collected and expanded to make a CancerStemCell cell line. In other embodiments, CancerStemCells selected from different selection processes are pooled together to make a CancerStemCell preparation in a disease-specific or species-specific manner. For example, CancerStemCell preparations specific for human breast cancer, human lung carcinoma, or mouse colon carcinoma were prepared and useful for research and drug development on particular cancer types.

Depending on cell source (a cancer cell line or tumor), cancer type, the cell density, nutrient amount (volume and concentration), serum level, size of the container, and other conditions such as the speed of shaking for a suspension culture, density of attachment factors on an attachment plate, and temperature fluctuations, the duration of each phase in the growth, death and survival curve may be varied (FIG. 2). The length of the lag phase may range from a few hours to many days. The lag phase is longer when the growing environment is more different than the previous growing environment. The log phase may range from days to weeks depending mostly on the cell types, initial cell number and the supply of nutrients. The stationary phase lasts days to weeks. The death phase lasts a few days to weeks. The cancer stem cell survival phase may last several days to weeks. Depending on the richness of cancer stem cells, it takes about 4-9 months to concentrate enough cancer stem cells for a successful cancer stem cell product.

Cancer stem cell products have been successfully developed for the following cancer types and species: CSC-185 for human lung carcinoma, CSC-221 for human colorectal adenocarcinoma, CSC-441 for human kidney rhabdoid tumor (Wilms' tumor), CSC-002 for human cervical adenocarcinoma, CSC-199 for human lung epidermoid carcinoma, CSC-022 for human breast adenocarcinoma, CSC-420 for human pancreatic carcinoma, CSC-480 for human colorectal adenocarcinoma, CSC-014 for human glioblastoma, CSC-130 for canine bone osteosarcoma, CSC-242 for guinea pig colorectal adenocarcinoma, CSC-200 for rat gliosarcoma, CSC-743 for rat ammary gland adenocarcinoma, CSC-600 for rat hepatoma, CSC-376 for rat prostate malignant carcinoma, CSC-043 for rat testis Leydig cell tumor, CSC-754 for rat neuroblastoma, CSC-539 for mouse mammary gland tumor, CSC-016 for mouse melanoma, CSC-026 for mouse liver hepatoma, CSC-642 for mouse Lewis lung carcinoma, CSC-638 for mouse colon carcinoma, CSC-142 for mouse renal adenocarcinoma, CSC-049 for hamster skin melanoma.

Still under development, cancer stem cell products may be made for the following: human placenta choriocarcinoma, human lung squamous cell carcinoma, human lung small cell carcinoma, human liver hepatocellular carcinoma, human skin malignant melanoma, human ovary adenocarcinoma, human thyroid squamous cell carcinoma, human brain neuroblastoma, human eye retina retinoblastoma, human prostate adenocarcinoma, human urinary bladder carcinoma, human bone osteosarcoma, human stomach gastric adenocarcinoma, human uterus uterine sarcoma, rat insulinoma, rat pituitary anterior adenoma, rat bone osteosarcoma, rat small intestine adenocarcinoma, rat urinary bladder tumor, mouse pancreas adenocarcinoma, and mouse rectum polyploid carcinoma

However, cancer stem cells were not found in the following cancer cell lines: CRL-2025™ for rat insulinoma, CRL-2118™ for chicken haptocellular carcinoma and CRL-2112™ for chicken bursa lymphoma. Tumors or other cell line sources may be needed to found cancer stem cells for these cancer types in the rat and chicken species.

The present invention further provides a whole-cell cancer vaccine derived from the CancerStemCells of the invention. Cancer vaccines are harmless variants or derivatives of cancer cells that preserve cancer specific antigens and can elicit immune responses against the cancer cells. Cancer vaccines do not grow into cancer cells in the culture or inside an individual. Compared to radiotherapy and chemotherapy, cancer vaccines are very safe to use as they elicit immune responses specifically targeting cancer cells, but do no harm to normal cells or tissues. Cancer vaccines derived from CancerStemCells have additional advantages because CancerStemCells are the rare but key players underlying cancer metastasis, drug resistance, and cancer relapse. Targeting the CancerStemCells in vivo will provide a promising means to eradicate cancer by its root. Since CancerStemCells accumulate a broad range of mutations which are shared with other types of cancer cells, immune responses elicited by CancerStemCell vaccine can kill not only CancerStemCells, but also mature cancer cells, pre-cancer cells, and metastatic cancer cells. In a xenograft tumor model, cancer vaccines derived from mouse melanoma CancerStemCells were so effective at eliminating cancer cells that five out ten vaccinated mice are cancer free for their lifetime (up to 1.5 years), whereas all the unvaccinated mice died within 25 days after CancerStemCell injection.

In some embodiments of the invention, a whole-cell cancer vaccine is made from CancerStemCells inactivated and made harmless by an appropriate process. The appropriate process to inactivate CancerStemCells will disable the CancerStemCells to grow or replicate while keep cell membranes intact. The inactivating process include, but not limited to, one or a combination of operations such as proteinase digestion, chemical treatment (e.g. formalin treatment and phenol treatment), physical treatment (e.g. heat and freeze-thaw-freeze treatment), and irradiation (e.g. y-ray, x-ray, microwave, and UV treatment). In one preferred embodiment of the invention, effective cancer vaccines are made from CancerStemCells using proteinase digestion. Proteinases inactivate CancerStemCells by digesting extracellular proteins and extracellular portion of membrane proteins of the CancerStemCells. Such proteinases can be selected, for example, from proteinase K, carboxypeptidase B, elastase, plasmin, endoproteinase Glu-C, endoproteinase Asp-N, endoproteinase Lys-C, endoproteinase Arg-C, chymotrypsin, or carboxypeptidase Y, caspases, subtilisin BL, M-protease, thermitase, subtilisin Carlsberg, subtilisin Novo BPN′, subtilisin BPN′, selenosubtilisin, tonin, blood coagulation factor XA, rat mast cell protease II, kallikrein A, pronase, trypsin, and anhydro-trypsin.

In some embodiments of the invention, cancer vaccines are derived from human CancerStemCells selected from a patient's tumor cells. The cancer vaccine can be used to treat the same patient to elicit immune responses to eradicate cancer cells.

In some embodiments of the invention, cancer vaccines are derived from cancer type-specific CancerStemCells. It is speculated that such cancer vaccines will have broad coverage of antigens specific for the particular cancer type, and can be used to cure the cancer in different patients.

In some embodiments of the invention, cancer vaccines are derived from a combined preparation of human CancerStemCells selected from cells of all the known human cancer types, thus making a pan-cancer vaccine targeting multiple human cancers. The antigen species in the pan-cancer vaccine may be diversified enough to potentially cover all antigen species in any human cancer. It is speculated that this pan-cancer vaccine can be used as a therapeutic as well as a preventive agent against different cancers.

Cancer stem cells are treated with Tumorase™ Cancer Vaccine Kit to make corresponding cancer vaccines (CV) including but not limited to: CV-185 for human lung caracinoma, CV-221 for human colorectal adenocarcinoma, CV-441 for human kidney rhabdoid tumor (Wilms' tumor), CV-002 for human cervical adenocarcinoma, CV-199 for human lung epidermoid carcinoma, CV-022 for human breast adenocarcinoma, CV-420 for human pancreatic carcinoma, CV-480 for human colorectal adenocarcinoma, CV-014 for human glioblastoma, CV-130 for canine bone osteosarcoma, CV-242 for guinea pig colorectal adenocarcinoma, CV-200 for rat gliosarcoma, CV-743 for rat ammary gland adenocarcinoma, CV-600 for rat hepatoma, CV-376 for rat prostate malignant carcinoma, CV-043 for rat testis Leydig cell tumor, CV-754 for rat neuroblastoma, CV-539 for mouse mammary gland tumor, CV-016 for mouse melanoma, CV-026 for mouse liver hepatoma, CV-642 for mouse Lewis lung carcinoma, CV-638 for mouse colon carcinoma, CV-142 for mouse renal adenocarcinoma, CV-049 for hamster skin melanoma.

### EXAMPLES

### Example 1

### Method for Making CancerStemCells from a Mouse Melanoma Cancer Cell Line

A mouse melanoma tumor cell line (CRL-6475, ATCC, Manassas, Va.) was cultured in flasks containing 60 ml medium such as Eagle's Minimum Essential Medium (30-2003, ATCC, Manassas, Va.) or RPMI-1640 Medium (30-2001, ATCC, Manassas, Va.) or F-12K Medium (30-2004, ATCC, Manassas, Va.) or Dulbecco's Modified Eagle's Medium (30-2002, ATCC, Manassas, Va.) or Leibovitz's L15 Medium (30-2008) with 5% fetal bovine serum USDA Premium (9871-5200, USA Scientific, Ocala, Fla.) at 37° C., 95% air, 5% CO₂. The CRL-6475 cancer cells were allowed to grow until confluency, and were kept in the same medium for a sustained period of time when the nutrients became depleted. Non-CancerStemCell cancer cells form a mono-layer of cells attached to the bottom of the culturing flask. When the nutrients in the medium became more and more depleted, the mono-layered non-CancerStemCells started to die out. The dead cells were detached from the bottom of the flask and could be easily separated from the attached cells. The duration of the death phase of non-CancerStemCells varies from several days to several weeks, depending on the cancer type, cell source, amount of nutrients, serum level, cell density and other conditions.

The CancerStemCells grew into multiple layers of cells instead of a mono-layer in a complete growth medium. The CancerStemCells are genetically mutated so that they are much more resistant to adverse effects of extreme culturing conditions than the non-CancerStemCells. During the prolonged period of nutrient depletion, rare spots of multiple layers of CancerStemCells survived and remained attached to the bottom of the culturing flask while the non-CancerStemCells eventually died out. The dead, detached cells were separated from the living CancerStemCells when the “old medium” was replaced with a fresh medium at the end of the death phase. When cultured in a complete growth medium, the survived CancerStemCells grew into islands of 3-dimensional, multilayer tumor spheres (see FIGS. 3A and 3B). When the flask was full of large islands of tumor spheres of CancerStemCells, the CancerStemCells were moderately digested by 0.25% 1× Trypsin (Life Technologies, Inc., Carlsbad, Calif.) and subcultured in a complete medium with serum.

Unlike the non-CancerStemCell peers which grow to form a mono-layer of cells, the subcultured CancerStemCells formed multilayer 3D in vitro tumor structures attached to the plate when cultured in a complete medium with serum. CancerStemCells grow in a three-dimensional manner, that is, they not only grow to expand in the 2D planar of the culture dish, but also grow multiple layers of cells stacking on one another (FIG. 3). Given sufficient nutrients, the CancerStemCells grew into three-dimensional tumor-like structures with seven to eight or even more layers of cells. The shape of the in vitro tumors is mostly hemispherical or other irregular shapes.

After generations of passage and subculture, the CancerStemCells can still maintain the sphere-forming ability indicating that the sphere-forming ability is due to genetic mutations in the genome, not temporary responses to external stresses. The in vitro 3D-tumors formed by the CancerStemCells of the invention are very similar to the early stage tumor formation in the body, providing an invaluable in vitro tumor model for cancer research, cancer drug screening, cancer diagnostic tests, and development of novel cancer therapy.

The sphere-forming CancerStemCells showed very strong in vivo tumorigenic potential tested by mouse xenograft experiments. 300, 3000, and 30,000 CancerStemCells were subcutaneously injected (sub-Q injected) into immune-deficient nude mice, and palpable tumors were found in 50%, 90%, and 100% of injected mice, respectively. It generally takes more than one million non-CancerStemCell cancer cells to produce tumors in a mouse. For CancerStemCells, as low as 300 cells can produce palpable tumors in the mouse indicating strong tumorigenic ability of these CancerStemCells.

The CancerStemCells enriched by the method above are tumorigenic, heterogeneous, and prone to mutation. They are selected by their ability to survive difficult culturing conditions and the ability to form multilayered 3D-tumor in vitro. Since they are not selected based on particular biomarkers, the population of the CancerStemCells could encompass a broad range of cells with a broad spectrum of mutations, which make them desirable for production of cancer vaccines bearing rich mutation information. CancerStemCells from different selection processes, including processes using different media, different extreme growth conditions, different cell sources (e.g. cells from cancer cell lines or tumors), are proportionately combined to make one cancer stem cell product that have a more broad coverage of cancer antigens (see FIG. 5).

### Example 2

### Whole-Cell Cancer Vaccine Made from CancerStemCells

Tumorase™ Cancer Vaccine Kit (TK-110, BioMedicure, San Diego, Calif.) was used to harvest CancerStemCells to make a cancer vaccine according to manufacturer's instructions. Briefly, 15-20 million melanoma CancerStemCells (CSC 016, BioMedcure, San Diego, Calif.) cultured in a 75 cm² flask were incubated with 3-5 ml of 0.25% 1× Trypsin (Life Technologies, Inc., Carlsbad, Calif.) to disassociate the cells from the flask. The disassociated cells were spinned down by centrifugation and suspended in a Tumorase™ solution (comprising proteinase K) at 10 million cells/ml. The CancerStemCells were incubated with Tumorase™ for 15 to 30 minutes at 37° C. until all the cells were separated and became individual giant round-shaped “liposomes” without attaching to each other. The resulting giant liposomal analogs were whole cell cancer vaccines, which were dead CancerStemCells with intact cell members.

About 300,000 giant liposomal analogs in the melanoma cancer vaccine (CV-016, BioMedicure, San Diego, Calif.) were used to vaccinate wild-type mice (c-57, 5 males, 5 females) on day 1 and day 15. On the 30^{th} day, 300,000 living CSC 016 melanoma CancerStemCells were sub-Q injected to 10 vaccinated and 10 unvaccinated mice. CSC 016 CancerStemCells quickly generated large tumors in 10 out of 10 unvaccinated mice and all the unvaccinated mice died within 25 days. The lower panel of FIG. 4 shows five unvaccinated mice with large tumors. None of the vaccinated mice developed any tumor within the first two months after the CSC 016 cells injection. Five vaccinated mice developed tumors between two and four months after the CSC 016 cells injection. The tumors developed in vaccinated mice were solid tumors that tended to stay local and non-mobile, whereas tumors developed in unvaccinated mice were soft and movable tumors that had high tendency to move to remote locations. Remarkably, five out of ten vaccinated mice were tumor free for the rest of their lifetime, which were shown in FIG. 4, upper panel. These data demonstrated that the cancer vaccine made from the CancerStemCells is so effective at eliciting immune responses against tumor cells that all the tumor cells can be eradicated by the immune system. The key to successfully kill every single tumor cells in the vaccinated mice lies at killing every tumor-initiating CancerStemCell.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

We claim:

1. An enriched preparation of CancerStemCells, which is selected from a population of cancer cells by eliminating non-CancerStemCells in the population under one or a combination of extreme culture conditions.

2. The CancerStemCells of claim 1, wherein the CancerStemCells form in vitro 3D-tumors.

3. The CancerStemCells of claim 1, wherein the CancerStemCells are tumorigenic.

4. In the claim 1, wherein the extreme culture conditions comprise at least one culture condition selected from the group consisting of: culturing under low nutrients/starvation, low oxygen level/hypoxia, low carbon dioxide level, and irradiation.

5. In the claim 1, wherein the extreme culture conditions comprise culturing in a medium with limited nutrients.

6. In the claim 1, wherein the population of cancer cells are obtained from a cancer cell line, a tumor, a bone marrow sample, or a blood sample with cancerous cells.

7. A CancerStemCell cell line made from the CancerStemCells of claim 1.

8. A combined preparation of claim 1, wherein CancerStemCells selected from different selection processes are pooled together to make the combined preparation of CancerStemCells.

9. A method of enriching CancerStemCells, comprising the steps of:
a, obtaining a population of cancer cells;
b, culturing the cancer cells in a growth medium;
c, eliminating non-CancerStemCells in the population under one or a combination of extreme culture conditions;
d, selecting cells surviving the extreme culture conditions, wherein the surviving cells are CancerStemCells;

10. The method of claim 9, wherein the CancerStemCells form in vitro 3D-tumors.

11. The method of claim 9, wherein the extreme conditions comprise at least one culture condition selected from the group consisting of: culturing under low nutrients/starvation, low oxygen level/hypoxia, low carbon dioxide level, and irradiation.

12. The method of claim 9, wherein the extreme culture conditions comprise culturing in a medium with limited nutrients.

13. The method of claim 9, further comprising a step of expanding the CancerStemCells in a complete medium.

14. The method of claim 9, further comprising a step of expanding a single CancerStemCell sphere to make a CancerStemCell cell line.

15. A cancer vaccine, which is a derivative of the CancerStemCells of claim 1 that are inactivated by an appropriate process.

16. The cancer vaccine of claim 15, wherein the CancerStemCells are inactivated by a protease treatment.

17. The cancer vaccine of claim 16, wherein the protease is Protease K.
